(19) United States
(12) Patent Application Publication
(10) Pub. No.: US 2021/0219840 A1
(43) Pub. Date: Jul. 22, 2021

(54) FLEXIBLE AND ADHESIVE ELECTRONIC DETECTION DEVICE THERMOMETER, CAPABLE OF MEASURING TEMPERATURE, STORING IT AND TRANSFERRING IT USING STANDARD NFC

(71) Applicant: Paolo MARINI, Arce (FR) (IT)

(72) Inventor: Paolo MARINI, Arce (FR) (IT)

(21) Appl. No.: 17/051,513

(22) PCT Filed: May 2, 2019
(86) PCT No.: PCT/IB2019/053582

§ 371 Date: Oct. 29, 2020

(30) Foreign Application Priority Data
May 7, 2018 (IT) 102018000005112

(51) Int. Cl. A61B 5/00; A61B 5/01; H04B 5/00

(52) U.S. Cl. CPC A61B 5/0008; A61B 5/01; A61B 2560/0214; H04B 5/0037; A61B 5/6833; H04B 5/0031

(57) ABSTRACT

Disclosed is a detector device for measuring and displaying clinical values in instantaneous mode. It is obtained from the printing of a sensor (on a thin rigid plastic support substrate, from here with the sensor constrained to the substrate, once the detector is positioned on the skin the device is characterized by elasticity and elongation. The clinical parameter detector includes: a rigid plastic support, a mechanism for acquiring, processing and transmitting the readings taken in digital mode, an adhesive component that fixes the acquisition, processing and transmission device to the rigid plastic support and an external patch-like component. An antenna is provided that allows the device to detect clinical values to interface via a wireless NFC connection with an external display device. This wireless NFC connection provides power to the detector device.

### FIELD OF THE INVENTION

The present invention finds application in the medical and pharmaceutical fields. It generally relates to a digital electronic detection system of clinical body parameters for medical use. More specifically, it relates to an adhesive detection device for medical use, which allows the measurement of clinical body values, such as temperature, instantly and without the use of a battery. Power is supplied to the device via the NFC standard.

### PRIOR ART

In the current scenario of clinical sensors for medical use and specifically concerning the measurement of body temperature, there are the following measuring instruments:
Measurement with liquid detection thermometer device
Measurement with digital detection thermometer device
Measurement with infrared detection thermometer device

All the instruments listed perform the measurement through more or less complex methods:
The liquid detection thermometer device uses the expansion of a fluid and a calibrated sheet
The electronic detection thermometer device uses thermoresistive sensors and more or less complex algorithms in order to reduce the measurement time
The infrared detection thermometer device uses the infrared spectrum in order to determine the temperature.

A clear drawback of the solutions according to the prior art is that liquid thermometers require sufficiently long measurement times starting from a minimum of 3 minutes. On the other hand, digital type thermometers require a very short measurement time, but not without measurement errors, since in order to accelerate the measurement time, the algorithm performs an estimate, which is subjected by its nature to an error. The measurement also affects the positioning of the instrument in the armpit or in the rectum, which in any case is not easy and therefore can be annoying for those who use it.

In infrared thermometers, the measurement may be subject to errors, since the instrument needs to be pointed correctly on the surface. In fact, pointing is not always easy to perform, especially in newborns, who move a lot.

Still in the field of infrared thermometers and in particular the models that detect fever after being inserted into the ear, the system is rapid but may not be completely reliable: the tympanic membrane can in fact be hot even in the absence of fever and this affects the reliability of the detection, or the presence of earwax in the duct can cause a lower temperature to be sensed. The cost of an infrared detection thermometer device is very high. In all the cases listed above, in order to be able to perform the measurement correctly, it is necessary to place the instrument on the area and wait for the time necessary for the system to perform the measurement, which can range from 2-3 seconds for infrared o a few minutes for digital.

Finally, all the instruments available on the market today require a power source, which can sometimes make these devices unusable when needed.

Other sensors for continuous monitoring of fundamental clinical parameters, such as blood glucose in diabetic subjects, are also known. These sensors are increasingly designed to support the person who is to be subjected to clinical checks. Recently the annoying solution of the prick to extract the drop of blood has been overcome and the new devices are characterized by a display that constantly communicates blood sugar levels, with alarms that warn in case of hypoglycemia or hyperglycemia; while more innovative devices allow sending glycemic values to small readers or directly to mobile apps.

These instruments have a fundamental drawback: the blood glucose sensor, which enters the epidermis from the outside, can easily come off with a little sweat or a bump. To address this problem, a solution has recently been devised with a subcutaneous implant of the sensor (a tube 1 centimeter long and 3 mm thick), through a cut on the arm under local anesthesia. On the skin surface, exactly above the sensor, the rechargeable transmitter is positioned, which sends alarms, warnings and notifications related to the glucose values on the app on the smartphone.

The advantage of this system is that if one sweats, dives or hits something, the most that can happen is that the receiver is disconnected, not the sensor, which is in a situation of absolute safety under the skin.

Moreover it is certainly a big advantage to finally be able to avoid the annoying pricks on the finger. In fact, applications, now tested as FreeStyle Libre, allow a complete picture of the to glycemic profile without pricking the finger.

The FreeStyle Libre kit consists of two parts:
a small sensor to wear and rest on the arm (it is s as big as a two-euro coin);
a reader that allows tracking of the glucose level in the patient's blood.

The small sensor does not have to be calibrated and has been designed to remain, attached to the arm, functioning for 14 days.

To detect the glycemic index, one just passes the reader over the sensor even above the clothes, and the data collected is displayed in a touch screen display.

This blood glucose meter without prick is, moreover, described in detail at the Abbott FreeStyle Libre link.

The solution according to the present invention makes use of completely different systems and technologies both in the positioning, in the operation of the detection device, and in the transmission of the acquired values.

The object of the present invention is to allow the implementation of a device for detecting clinical parameters that has the ability to adapt to the most complex shapes. The sensor is in fact printed on thin rigid or silicone plastics, which give it flexibility without loss of performance depending on the field of use.

A further object of the present invention is to provide a detector of clinical bodily parameters that can be bent, lengthened or twisted, without losing its functionality in any way.

It is a further object of the present invention to provide a detector of clinical body parameters that does not exhibit any error due to positioning or alignment, since it is constrained to the measurement surface by an adhesive. In fact, we want the detector to be produced separately from the adhesive, making it reusable.

A further object of the present invention is to provide a detector of clinical parameters with multiple, immediate measurements, without the need to prepare the instrument for measurement. The invention provides to operate with a detector of clinical values which stores the energy transmitted by the transmission with the NFC protocol and charges a capacitor, which allows measurements to be made at regular time intervals. The number of measurements will depend on the type of capacitor used.

Finally, it is an object of the present invention to provide a detector of clinical body parameters that uses acquisition protocols, processing data of the acquired data among the most widespread and recognized as standard in the field of medical physics, in order to make the embodiment of the invention immediate, reliable and easy to use for the end user.

The objects set out above are obtained by means of a clinical adhesive detector, for medical use, which allows the clinical parameter to be measured instantaneously and without the use of a battery. Power is supplied to the device via the NFC standard. The detector of clinical body parameters is elastic and deformable, since it is printed on plastic material (Kapton) which makes it particularly suitable for the purpose of the application.

Furthermore, the detector of clinical bodily parameters is adhesivized, in order to adhere perfectly to the skin for several days. In fact the detector can be folded, rolled and stretched, without ever losing its functional features.

An element that characterizes the versatility of the invention is that the detector of clinical parameters can be printed on any other plastic polymer as long as it complies with the regulations in force in the medical field.

Moreover, the invention is characterized by a series of distinctive elements as it is referred to in claims 1 to 11.

For the sole purpose of better clarifying the invention and without thereby limiting the scope and the sectors in which it can be applied, some particular embodiments will be described below, with reference also to the accompanying drawings.

### BRIEF DESCRIPTION OF THE DRAWINGS

FIG. 1 is a schematic representation of a first transversal view of the detector according to the present invention;

FIG. 2 is a top plan view of the detector of clinical parameters according to the invention;

FIG. 3 is a further transversal view of the detector according to the invention;

FIG. 4 is a plan view of the detector showing the hole map obtained on the substrate;

FIG. 5 is a visual representation of the flexibility features of the detector according to the present invention;

FIG. 6 is a general block diagram of the detector according to the present invention;

FIG. 7 is a graph relating to the evaluation of the tensile strength of a substrate without holes for a detector device according to the invention;

FIG. 8 is a graph relating to the evaluation of the tensile strength of a substrate with a matrix of holes for a detector device according to the invention.

The detector of clinical bodily parameters 10 functions as an adhesive thermometer for medical use, which allows the measurement of body temperature, instantly and without the use of a battery. Power is supplied to the device via the NFC standard. The detector of clinical body parameters is elastic and deformable, since it is printed on plastic material 1, Kapton, which makes it particularly suitable for the purpose of the application.

In order to adhere perfectly to the skin, the clinical parameter detector 10 is coated. It can be folded, rolled and stretched, without ever losing its functional features. So the detection device thermometer thus created has the ability to adapt to the most complex shapes. The sensor 4 is in fact printed on thin rigid or silicone plastics, which give it flexibility without loss of performance depending on the field of use.

Furthermore, the detector of clinical bodily parameters can be printed on any other plastic polymer 1 as long as it is an insulator and complies with the regulations in force in the medical field.

The detector of clinical body parameters according to the invention is very thin, having a thickness of 925 um (micrometers) and has a weight of just 0.527 g (the measurement was performed with the precision scale Ohaus Adventurer Pro). Depending on the chosen adhesive, the weight of the detector of clinical body parameters can increase or decrease significantly.

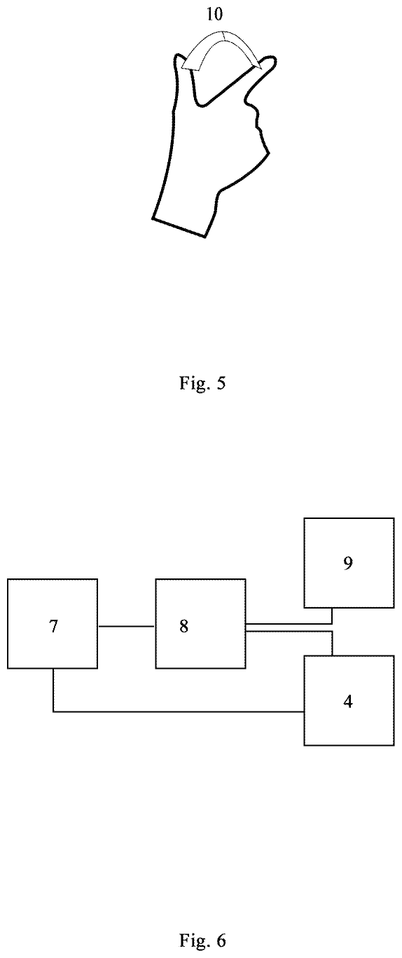

As can be seen in the images in FIG. 5, the surface can be deformed without undergoing any electrical or functional alteration.

In this context, the meaning of the term “rigid plastic substrate” must be clarified. The adjective “rigid” must be attributed first of all to the properties of the support material which are inherent in maintaining the starting dimensions in which the detection device 10 is initially constituted. In fact, although subjected to various mechanical stresses such as compression, traction or bending, in any case the initial dimensions are maintained.

On the other hand, the properties inherent in the flexibility of the support are also excellent, since the same substrate can be subjected to traction or bending without this involving any permanent deformation.

In summary, the material, of a basically plastic type, is able to withstand mechanical stresses of different types, however returning to its original shape and dimensions. So the term “plastic” should not be understood as a plastic container or a plaster-like layer that compresses, becomes a compact mass in a ball, but only gives an indication of the flexibility and elasticity of the substrate itself.

The clinical parameter detector 10 requires a measurement time of only 100 msec (milliseconds) for the execution of a measurement. However, a distinction should be made of the measurement time which the clinical parameter detector needs to reach its thermal inertia, when it is placed the first time. In fact, on first use, the clinical parameter detector 10 needs on average 5 seconds to reach its thermal inertia, after which it faithfully and instantaneously follows the body temperature, returning an instantaneous measurement. No error due to positioning or alignment is attributable to the clinical parameter detector 10, since it is constrained to the measurement surface by an adhesive 2. Furthermore the sensor 4 can be produced separately from the adhesive 2, making it reusable.

The energy transmitted in the NFC communication is stored by the sensor and charges a small capacitor 6, which allows it to perform measurements at regular times. The number of measurements depends on the type of capacitor used. The data of the detected temperature are acquired in a memory area, which can be read by any device operating with the NCF protocol. With this configuration, multiple and immediate measurements can be performed, without the need to prepare the instrument for measurement.

The antenna 7, responsible for communications according to the NCF standard, is optimized to maximize the energy transmitted to the detector of clinical parameters even when there is no perfect alignment with respect to the plane integral with the antenna.

The detection device thermometer 10 can have different shapes including: circular, triangular, square, rhomboid, elliptical, rectangular. It will be shown below that a form associated with the substrate with both longitudinal and transversal symmetry axes is fundamental.

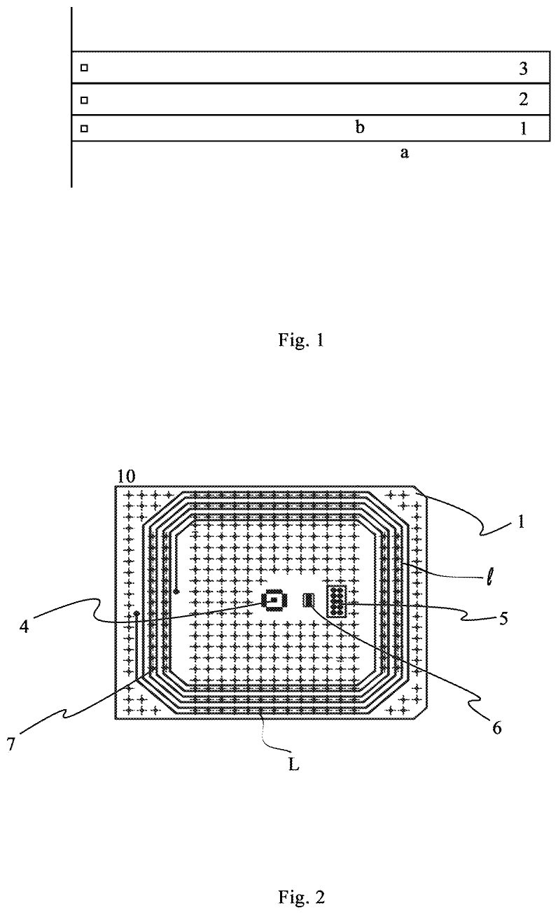

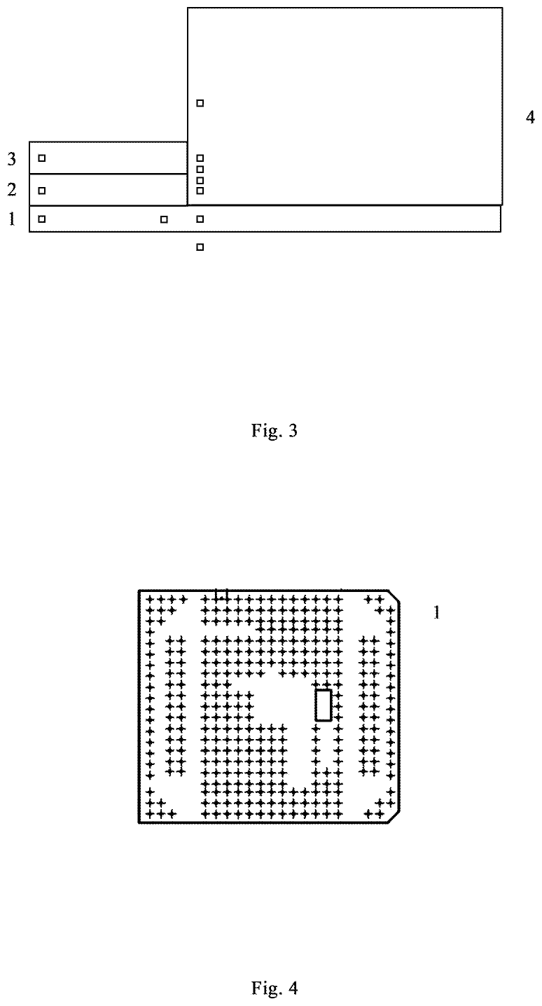

As can be seen in FIG. 1 and FIG. 2, the clinical parameter detector 10 consists of the following parts:
Support of Kapton 1
Adhesive 2
Patch 3
Antenna 7
CPU (Microcontroller) 4
Programming Connector 5
Decoupling and power supply capacitor 6

The individual components are analyzed below.

The Kapton support 1 is commonly used in electronics only for the purpose of producing flexible printed circuits, the direct use thereof for making biometric thermometer detectors is certainly not known. In this case, this support was selected in order to implement the subject application, since Kapton has flexibility properties and, for small thicknesses, low thermal resistance.

Experimental tests have shown that the thicknesses of the support that guarantee the best performance, in terms of mechanical stability and thermal conductivity, range from 0.025 mm to 0.125 mm. A value of 0.075 mm was chosen for the prototype, as it has low market costs and guarantees excellent flexibility and mechanical strength.

The patch 3 is rectangular in shape and has dimensions of 59.69×53.34 mm. In fact, it consists of a cloth tape with an adhesive applied exactly like in a classic plaster.

The size of the clinical parameter detector 10 may range from 15×15 mm to the maximum size of 65×65 mm.

The thickness of the clinical parameter detector 10 ranges from 0.925 mm to 1.5 mm, depending on the type of adhesive used. The maximum size is reached by the processor which is currently in a QFN format package. By choosing the drop package, the thickness of the detector of clinical body parameters can go down to 0.575 mm at its highest point.

As can be seen in FIG. 4, the support 1 has a series of holes arranged in a matrix on the entire circuit. This choice has a triple purpose:
Minimizing the mass of the detector 10.
Allowing the natural transpiration of the skin.
Imparting elasticity and flexibility to the substrate 1 itself.

From FIG. 4 it is clear that the holes are arranged in a matrix but areas are left free of holes longitudinally at the components and the path of the antenna, so as not to weaken the support structure 1. The space between the holes is 2.54 mm. The holes may have a variable diameter of between 0.5 mm and 1.5 mm depending on the thickness of the support 1 and of the adhesive 2 in order to be able to reach the above mentioned weights.

Clearly, the smaller the mass, the faster the thermal inertia is reached. In the present case, the clinical body parameter detector has a weight that can fluctuate between 0.4 g and 1.5 g depending on the thickness of the materials chosen.

In order to show that the Kapton has all the features described above, it was shown that under the same conditions (environmental parameters, sensor positioning and clothing), the temperature determined by the sensor corresponds to a repeatable objective measurement. To this end, the following premises are reported:
As is known, clothing reduces energy loss from the human body and is therefore classified according to the level of thermal insulation provided. The unit of measurement usually used for measuring the surface thermal resistance in the clothing field is the clo.
The conversion between clo and SI (m² K /W) is performed by the following relationship:

1lclo=0,155^{m}^{²}^{K}/W

The following table shows the typical thermal resistances of commonly used clothing:

TABLE 1
Typical values of clothing thermal resistance

| | | | | | Thermal |
|---|---|---|---|---|---|
| | | | Iclu | Surface | Resistance |
| Description of the garments | | Iclu(clo) | m2K/W | (4 × 4 mm) | K/W |
| Linen, | Stockings | 0.02 | 0.003100 | 0.0000016 | 1,937.50 |
| lower part | Panties | 0.03 | 0.004650 | 0.0000016 | 2,906.25 |
| of the body | Boxer | 0.04 | 0.006200 | 0.0000016 | 3,875.00 |
| | Knee-length wool | 0.06 | 0.009300 | 0.0000016 | 5,812.50 |
| | underpants | | | | |
| | Ankle-length underpants | 0.10 | 0.015500 | 0.0000016 | 9,687.50 |
| Linen, | Bra | 0.01 | 0.001550 | 0.0000016 | 968.75 |
| upper body | Sleeveless shirt | 0.06 | 0.009300 | 0.0000016 | 5,812.50 |
| | T-shirt | 0.09 | 0.013950 | 0.0000016 | 8,718.75 |
| | Long sleeved shirt | 0.12 | 0.018600 | 0.0000016 | 11,625.00 |
| | Nylon slip | 0.14 | 0.021700 | 0.0000016 | 13,562.50 |
| Shirts- | Tops | 0.06 | 0.009300 | 0.0000016 | 5,812.50 |
| Blouses | Short-sleeved blouse | 0.09 | 0.013950 | 0.0000016 | 8,718.75 |
| | Lightweight long- | 0.15 | 0.023250 | 0.0000016 | 14,531.25 |
| | sleeved blouse | | | | |
| | Lightweight long- | 0.20 | 0.031000 | 0.0000016 | 19,375.00 |
| | sleeved shirt | | | | |
| | Normal long-sleeved | 0.25 | 0.038750 | 0.0000016 | 24,218.75 |
| | shirt | | | | |
| | Flannel long-sleeved | 0.30 | 0.046500 | 0.0000016 | 29,062.50 |
| | shirt | | | | |
| | Long-sleeved blouse | 0.34 | 0.052700 | 0.0000016 | 32,937.50 |
| | with high collar | | | | |
| Trousers | Shorts | 0.06 | 0.009300 | 0.0000016 | 5,812.50 |
| | Walking shorts | 0.11 | 0.017050 | 0.0000016 | 10,656.25 |
| | Light trousers | 0.20 | 0.031000 | 0.0000016 | 19,375.00 |
| | Normal trousers | 0.25 | 0.038750 | 0.0000016 | 24,218.75 |
| | Flannel trousers | 0.28 | 0.043400 | 0.0000016 | 27,125.00 |
| | Suit | 0.28 | 0.043400 | 0.0000016 | 27,125.00 |
| Overalls | Daily suit with belt | 0.49 | 0.075950 | 0.0000016 | 47,468.75 |
| | Overalls | 0.50 | 0.077500 | 0.0000016 | 48,437.50 |
| Highly | Multi-component suit | 1.03 | 0.159650 | 0.0000016 | 99,781.25 |
| insulating | “Fiber pelt” suit | 1.13 | 0.175150 | 0.0000016 | 109,468.75 |
| jumpsuits | | | | | |
| Sweaters | Vests | 0.12 | 0.018600 | 0.0000016 | 11,625.00 |
| | Light sweater | 0.20 | 0.031000 | 0.0000016 | 19,375.00 |
| | Long-sleeved sweater, | 0.26 | 0.040300 | 0.0000016 | 25,187.50 |
| | light, with | | | | |
| | high collar | | | | |
| | Classic sweater | 0.28 | 0.043400 | 0.0000016 | 27,125.00 |
| | Heavy sweater | 0.35 | 0.054250 | 0.0000016 | 33,906.25 |
| | Heavy high collar | 0.37 | 0.057350 | 0.0000016 | 35,843.75 |
| | sweater | | | | |
| Jackets | Waistcoat | 0.13 | 0.020150 | 0.0000016 | 12,593.75 |
| | Light summer jacket | 0.25 | 0.038750 | 0.0000016 | 24,218.75 |
| | Jacket | 0.35 | 0.054250 | 0.0000016 | 33,906.25 |
| | Gown | 0.30 | 0.046500 | 0.0000016 | 29,062.50 |
| Outdoor | Coat | 0.60 | 0.093000 | 0.0000016 | 58,125.00 |
| clothing | Under-jacket | 0.55 | 0.085250 | 0.0000016 | 53,281.25 |
| | Parka | 0.70 | 0.108500 | 0.0000016 | 67,812.50 |
| | Duster in several parts | 0.52 | 0.080600 | 0.0000016 | 50,375.00 |
| Miscellaneous | Socks | 0.02 | 0.003100 | 0.0000016 | 1,937.50 |
| | Thin ankle socks | 0.05 | 0.007750 | 0.0000016 | 4,843.75 |
| | Long thin socks | 0.10 | 0.015500 | 0.0000016 | 9,687.50 |
| | Nylon stockings | 0.03 | 0.004650 | 0.0000016 | 2,906.25 |
| | Shoes (thin sole) | 0.02 | 0.003100 | 0.0000016 | 1,937.50 |
| | Shoes (thick sole) | 0.04 | 0.006200 | 0.0000016 | 3,875.00 |
| | Boots | 0.10 | 0.015500 | 0.0000016 | 9,687.50 |
| | Gloves | 0.05 | 0.007750 | 0.0000016 | 4,843.75 |
| Skirts, | Miniskirt, 15 cm above | 0.10 | 0.015500 | 0.0000016 | 9,687.50 |
| dresses | the knee | | | | |
| | Lightweight skirt, 15 cm | 0.18 | 0.027900 | 0.0000016 | 17,437.50 |
| | below the knee | | | | |
| | Heavy skirt (winter) | 0.25 | 0.038750 | 0.0000016 | 24,218.75 |
| | Lightweight, sleeveless | 0.25 | 0.038750 | 0.0000016 | 24,218.75 |
| | dress | | | | |
| | Winter dress, long | 0.40 | 0.062000 | 0.0000016 | 38,750.00 |
| | sleeves | | | | |
| Night | Long robe | 0.30 | 0.046500 | 0.0000016 | 29,062.50 |
| dresses | Lightweight dressing | 0.15 | 0.023250 | 0.0000016 | 14,531.25 |
| | gown with belt | | | | |
| | Hospital dressing gown | 0.31 | 0.048050 | 0.0000016 | 30,031.25 |
| | Long-sleeved pajamas | 0.50 | 0.077500 | 0.0000016 | 48,437.50 |
| | Pajamas covering the | 0.72 | 0.111600 | 0.0000016 | 69,750.00 |
| | feet | | | | |
| | Nightwear underpants | 0.10 | 0.015500 | 0.0000016 | 9,687.50 |
| Gown | Long sleeves, with a | 0.53 | 0.082150 | 0.0000016 | 51,343.75 |
| | long cape | | | | |
| | Long sleeves, with cape, | 0.41 | 0.063550 | 0.0000016 | 39,718.75 |
| | short | | | | |

Below is a table showing the typical Kapton insulation parameters as a function of thickness:

TABLE 2
List of thermal resistances for Kapton with
thermal conductivity equal to 0.12 W/mK

| Thickness [m] | Thermal resistance [K/W] |
|---|---|
| 0.0000075 | 3.90625 |
| 0.0000125 | 6.510416667 |
| 0.0000250 | 13.02083333 |
| 0.0000500 | 26.04166667 |
| 0.0000750 | 39.0625 |
| 0.0001250 | 65.10416667 |

The table in question shows typical values for a thermal conductivity value equal to 0.12 W/mK, but it should be said that these values can oscillate between 0.1 and 0.4, increasing the resolution of the values in the field.

In order to be able to compare the data correctly, there is a need to consider a use case of the sensor with a clothing item. In order to demonstrate the duality of use of Kapton, a sleeveless undershirt is chosen as the garment. This is obviously a worst case scenario, since only one item of clothing is considered (without additional clothing, such as a shirt, jacket or shirt). For the undershirt, there is a thermal resistance of:

R_{Shirt}=5812,5 K/W

The sensor data sheet indicates a thermal resistance between junction and package equal to 19.6 K/W:

R_{Package}=19,6 K/W

If we assume the direct mounting of the sensor on the skin, the total thermal resistance is equal to:

R_{T}=R_{Shirt}+R_{Support}+R_{Package}

Indicating with T_{P} the skin temperature, with T_{S} the sensor temperature and with T_{A} the temperature of the external environment, we can synthesize the temperature of the sensor as follows:

$${T}_{S} = {T}_{P} ( 1 - \frac{{R}_{Package}}{{R}_{T}} ) + \frac{{R}_{Package}}{{R}_{T}} {T}_{A}$$

The measurement error due to the thermal resistances involved in the measurement method with direct use is:

$$Temperature Drop with direct mode \% = \frac{{T}_{P} - {T}_{s}}{{T}_{P}} \times 100 = \frac{{R}_{Package}}{{R}_{T}} \frac{{T}_{P} - {T}_{A}}{{T}_{P}} \times 100$$

If we assume a temperature of the measurand equal to T_{P}=37° C. and a room temperature of T_{A}=22° C., the following is obtained:

Temperature Drop %=0,14%

If it is now assumed that one is in the case of indirect mounting. In this case, the thermal resistance that the heat flow encounters between the surface of the skin and the sensor is equal to:

R_{S}=R_{Support}+R_{Package}+R_{Plaster}

In the case in question, it was not possible to obtain the data relating to the thermal features of the patch. However, in a more general use, there are adhesives that have negligible thermal resistance values with respect to R_{Support} and to R_{Package}. Therefore it is permissible to approximate the above relation as follows:

R_{S}=R_{Support}+R_{Package}

The total resistance is the same as for the previous case:

R_{T}=R_{Shirt}+R_{Support}+R_{Package}

It is therefore possible to provide the relation that indicates the temperature loss as a percentage of the actual value of the measurement:

$$Temperature Drop with indirect mode \% = \frac{{R}_{S} ( {T}_{P} - {T}_{A} )}{{R}_{T} {T}_{P}} \times 100$$

As in the previous case, we assume a temperature of the measurand equal to T_{P}=37° C. and a room temperature of T_{A}=22° C.:

Below are the tabulated data for different thicknesses of Kapton:

TABLE 3
Percentage temperature drop with respect to the absolute value

| Thickness | R_{Package} | R_{Support} | R_{T-shirt} | R_{S} | R_{T} | Temp. drop |
|---|---|---|---|---|---|---|
| 0.0000075 | 19.6 | 2.130681818 | 5812.5 | 21.7306818 | 5834.23068 | 0.148987471 |
| 0.0000125 | 19.6 | 3.551136364 | 5812.5 | 23.1511364 | 5835.65114 | 0.158687597 |
| 0.0000250 | 19.6 | 7.102272727 | 5812.5 | 26.7022727 | 5839.20227 | 0.182917265 |
| 0.0000500 | 19.6 | 14.20454545 | 5812.5 | 33.8045455 | 5846.30455 | 0.231288296 |
| 0.0000750 | 19.6 | 21.30681818 | 5812.5 | 40.9068182 | 5853.40682 | 0.279541945 |
| 0.0001250 | 19.6 | 35.51136364 | 5812.5 | 55.1113636 | 5867.61136 | 0.3756988 |

As can be seen in the table, by dimensioning the thickness of the Kapton, in an industrial mass production, it is possible to obtain indirect mode performances completely comparable to those of the direct mode. In fact the best choice goes to Kapton with a thickness of 0.0000075 m, which gives a percentage drop of 0.148%.

Therefore on the basis of the appropriate choice of the substrate dimensions, in the study carried out, it was mathematically demonstrated that the percentage variation in the measurement with sensor mounted on the skin side or with sensor mounted on the clothing side is minimal and the behavior from the point of view of the thermal resistance is the same. For a large-scale industrial production, in fact, the gap is of the order of a thousandth with respect to the order of magnitude of the measurement.

It should also be emphasized that both the mathematical study and the empirical tests show that the theoretical hypotheses are actually verified since, on the basis of the measurements performed, no significant changes were acquired in the behavior of the detectors 10. For this type of examination, Kapton supports were made in the laboratory with different width and length, measurements were performed both with the wide Kapton and with the narrow Kapton and it was possible to detect the same temperature measurement.

In reality, even modulating the dimensions of the Kapton in even wider ranges, significant variations could not have been detected as the differences were very small, practically around 0.008%. Even the measurement instruments usually used do not have a resolution so sensitive as to allow detection. That is to say that the test was always performed with Kapton of different sizes without detecting any difference in temperature measurements. In other words, the mathematical approach is supported by laboratory analysis.

It has been shown, with the formulas and with the tables 1, 2 and 3, that by reducing the thickness of the Kapton, the same identical performances are obtained, with an arrangement that provides the sensor positioned above or below the substrate.

It is evident that the results of the experimentation validate the preliminary theoretical examination. The range of values examined was wide: experimental tests were performed with Kapton with a thickness of 7 μm and Kapton with a thickness of 75 μm and in fact the result was the same.

In theoretical terms, if a smaller Kapton thickness is chosen, it is still equivalent to other larger thicknesses, and in fact both at an experimental and a mathematical level, using for example the 75 μm Kapton and placing it first on the front side and then on the back there is no appreciable change in the measurement result. In terms of measurement error, the order of magnitude of the error is maintained at a level of 0.1° C. Only the width of the substrate 1 influences the detection in relation to the parameters of the selected item of clothing, as foreseeable in relation to the different extension of the contact surface.

The test was carried out under the worst hypothetical conditions: assuming that you put yourself in the condition where the clothing consists only of a short-sleeved cotton shirt, the measuring instrument is positioned below this garment and the very high associated thermal resistance is detected.

The effects of the use of Kapton as a material for the substrate are therefore evident, as it already gives a measurement error of only 0.05° C. If a 70 μm Kapton is used, putting the sensor on the clothing side, this error even if in fact doubles from 0.14% to 0.28%, produces a minimum variation: this is an error of an order of magnitude so small that, ultimately, it affects only with a margin of +0.1° C. In case one wants to work with even more precise measurements, one will still work on the thickness of the Kapton to obtain results that lead to variations below 0.1° C. However, reference is made to preliminary choices to be implemented if a very fine measurement is desired with a final value that has a final precision below 0.1° C.

It is therefore necessary to reiterate the fundamental principle according to which, for very fine measurements, the choice of the thickness of the support is functional on the mounting side of the sensor. In fact the sensor can be put in contact with the skin either directly (sensor mounted on the wall that is in contact with the skin), or indirectly (sensor mounted on the face opposite to the contact surface with the skin).

In the cases described above, there are different needs in terms of thermal insulation. This means that if the contact with the skin is direct, it is preferable to have a support material with high thermal resistance. In the case in which the mounting is indirect, it is certainly preferable to use a support with low thermal resistance, since the thermal resistance of the support is interposed between the sensor and the human body.

The Kapton itself has a very high thermal resistance, but since it is produced with different thicknesses, it is then possible to intervene precisely on the sizing of the thermal resistance that is most convenient for the application, choosing an appropriate thickness. For these conditions of fine detection of the temperature, as stated there are two distinct operating conditions:
A-If the sensor 4 is mounted in direct contact “a” with the skin, the device will be sized with Kapton 1 which has a higher thermal resistance, so in this case it works as an insulator. Actually acting as an insulator, the temperature of the sensor 4 remains isolated near the skin. If the comparison is allowed, it is, in practice, the same behavior as the clothing worn, which never reaches body temperature precisely because it is characterized by a high insulation degree.
B-Instead, on the contrary, if the sensor is mounted on the other side outside the skin, on the other side “b” of the support, one is interested that the thermal energy is quickly accumulated. A reduced thickness of the Kapton support 1 is therefore required which allows a rapid passage of temperature towards the sensor 4. In this other case, the head of the sensor 4 is arranged in contact with the support and not with the skin.

In other words, Kapton, as is known, is not a good thermal conductor but in case one wants to work with a high sensitivity thermometer, surprisingly, it is well suited to act as both a good conductor and a good thermal insulator. This is due to the fact that Kapton is produced on very variable thicknesses, which allows it to be used in its dual capacity.

In the implementation of the invention, the choices made are a function of what has been described above, comparing the thermal resistance of the processor package chosen with the thermal resistance of the Kapton. The technical data sheet of the product shows a thermal resistance of the package equal to 19.6 K/W. Since the sensor was mounted on the skin side, the choice must be made on a Kapton with high thermal resistance, in order to keep the heat as isolated as possible from the rest of the system. The tests showed that a good compromise between performance and thickness was achieved with a Kapton with a thickness of 75 μm (micrometers), which has an equivalent resistance of 39.0625 K/W.

In the event that one chooses to mount the sensor on the side opposite to the surface of contact with the skin, the choice goes to a Kapton with low thermal resistance, in order to obtain a better and faster transfer of heat to the sensor body.

As stated, the pricking of the substrate 1 allows the same to obtain better performances in terms of tensile elasticity.

Elongation tests were performed on the sensor with and without holes. The following results refer to the maximum elongation in mm without the sensor being damaged or losing shape:

TABLE 4
Maximum deformation without loss of shape or functionality

| | Sensor with | Sensor without |
|---|---|---|
| | holes | holes |
| Lengthening on the short side | 6.42 mm | 2.10 mm |

In order to verify the improvements made by the pricks, tensile tests were performed on the device with and without holes. The tests were performed using a Controls model TRIAX50 press with SR-LTF load cell (s/n 170901) and Controls electrical measurement transducer, model 82-P0334 (s/n 04117780).

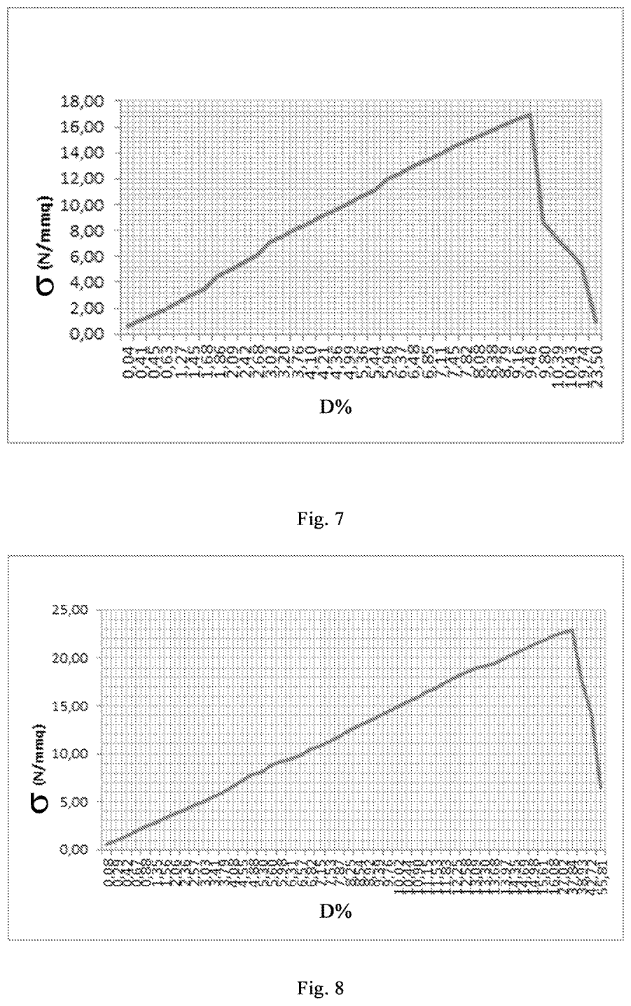

In FIGS. 7 and 8, the ordinate shows the rupture sigma in N/mm², as a function of the percentage deformation that appears on the abscissas. In these figures, the differences in the graphs relating to the evaluation of the tensile strength of a substrate without/with hole matrix for a detector device according to the invention are evident.

As one can easily see in the graph, the break point of the substrate without holes is for a unit load of σ=16,97 N/mm².

For the substrate with the holes, the breaking point is reached with a unitary load equal to σ=22,82 N/mm². This solution shows an increase in performance on the breaking load of 34.47%.

From the graphs, it is clear that even the elastic modulus undergoes a significant increase in performance.

If one places himself on the graph at a unit load of approximately σ=10 N/mm², we note that the elastic modulus behaves for the two specimens in the following way:

TABLE 5
Elastic Module for specimens with and without holes
Elastic Module at the unit load of σ = 10 N/mm²

| | |
|---|---|
| Substrate with holes | 153,22 N/mm² |
| Substrate without holes | 203,55 N/mm² |

As can be seen, the substrate with the holes has an elastic modulus lower by about 25.09%, which translates into a better propensity to stretch for the same effort.

The profile of the drilling basically takes an annular, quadrangular, concentric structure, thereby giving the flat structure the typical conformation whereby it is known that a hollow tube has a bending resistance that is greater than the resistance offered by the same solid tube. In fact, the conformation of the solid areas (without holes) as shown in FIG. 4, is that of a structure with several concentric interposed voids, thus assuming the evident bending resistance typical of laminar structures placed in series one to the other. In this case everything must be transposed to the level of elastic response, more particularly in relation to the condition of resistance to external stresses due to the extension and/or bending on the skin and therefore of elasticity which is confirmed by tables 4 and 5 and which is to be attributed to the amortization of tangential stresses at the same twisting moment. In fact, therefore, the substrate 1 with concentric ring-shaped matrix drilling is able to withstand a greater twisting moment with respect to a non-perforated substrate.

In practice, the configuration of the drilling alternating with solid areas, according to a series of concentric rectangles, is the result of the combination of different needs that lead to a single extremely advantageous result. Indeed, on the one hand there is the need to maintain non-perforated areas so as to be able to allocate active components (antenna, microprocessor, connector), on the other there is the need to give the substrate its own flexibility and resistance to traction. This dual objective is achieved with a single solution that reconciles the two requirements, the conformation of n rings, concentric squares, of holes alternating with solid areas, which nevertheless maintain a marked linearity along the two dimensions 1 and L typical of length and width of the rectangular substrate 1.

The antenna 7 has a spiral shape, with octagonal geometry. The dimensions, in this preferred embodiment, are 50.08×52.07 mm. The dimensions of the central octagon with irregular symmetry provide the measures of the long sides of 1 and L, respectively, of 50 mm and 52 mm and the measures of the short sides that range from 10, 5 mm for the outer side, to 5 mm for the side innermost side.

The antenna 7 is designed to maximize the power received on the plane integral with the antenna itself. This choice allows the sensor to be read even when there is no perfect alignment between the sensor and the receiving antenna. The result is very important, since in current NFC technologies, the antenna requires perfect alignment between the two transceiver antennas, making the reading subject to an alignment factor. This type of antenna, instead, allows the sensor to be read even when there is no perfect alignment between the antennas, both on the axis normal to the plane, and between the planes themselves.

The microcontroller 4 used is of the type: NHS3100. The choice is justified by the fact that this MCU has inside it all that is needed for the correct execution of the measurement, namely: an NFC interface, a high precision PTC sensor, a microprocessor. The choice is not binding, since any other processor can be used with the same features mentioned above.

In order to carry out a measurement with an error margin of +/−0.1° C., the sensor was calibrated in a thermal chamber with steps of one degree Celsius in the temperature bands comparable to the clinical area: 35˜42° C. In order to make the measurement accurate, calibrations were performed at 35° C., 36° C., 37° C., 38° C., 39° C., 40° C., 41° C., 42° C. This data allows you to perform a very fine data interpolation, so as to obtain the required accuracy.

The programming connector 5 has the sole purpose of transferring the compiled code inside the processor, but it is not the only programming method, since this can also occur through the same NFC protocol, in order to make the process fast in terms of industrialization.

The decoupling capacitor 6 has the task of decoupling the signal part from the power supply. The value can range from 100 nF to 100 uF and is functional to the sensor's operating time in the standby state. This means that the sensor can continue to perform sampling even after the charging step of the circuit, and return the sampled data to the next reading. The capacitor of the prototype made is of SMT 0805 type, but in order to make the component circuit-less, it may be printed on FCB.

The adhesive component 3 having the function of a patch used to make the clinical parameter detector adhere to the skin is the Soffix Stretch produced by PIC.

However, any adhesive may be used as long as it is for medical use.

The adhesive 2 (glue) used in order to make the sensor support adhere to patch 3 is LOCTITE 4601, but any other adhesive that has the same features may be used.

The thickness of the patch 3 is only 0.925 mm at the highest point. The support part measures 0.275 mm.

The functional logic block diagram of the body clinical parameter detector 10 is described below, as it is presented in FIG. 6. The functional components of the clinical parameter detector 10 are the following:
Antenna 7,
AC/DC Decoupler 8,
Temperature sensor 9,
Microcontroller 4.

This scheme highlights the fundamental features that are necessary in order to be able to monitor the temperature with the method according to the invention.

When the system is fed through the NFC antenna 7, the decoupling capacitor 8 stabilizes the supply voltage of the microcontroller 4. The latter makes the measurement through the sensor 9 (which can be either internal to the microcontroller or external), stores it in a memory area shared with the NFC transmitter. In order to perform the measurement, the data is transferred to a reader together with the calibration data. It will then be the task of the reader to perform a correct transformation of the data into temperature, with a simple linear interpolation of the calibration data.

The sensor 9 does not need battery power because it uses the power provided by NFC technology. In order to give an idea of the potential offered by the system, applications have been developed on Android mobile phones, which display and show the result on the screen. To make it work, simply place the phone near the clinical parameter detector 10. As soon as the measurement is performed (a few thousandths of a second), the phone emits a slight vibration, and shows the result of the measurement on the display.

In a preferred embodiment of the present invention, a DIE Hybrid assembly technique is used to manufacture the detector. In order to improve the general features of the acquisition, the sensor is mounted in DIE format with hybrid technique. This means that the processor is mounted on the circuit in its native form (i.e. the silicon wafer) directly on the support with wire bonding technique. Furthermore, with the same technique, the decoupling capacitor is placed near the processor. Everything is then electrically isolated through a drop of resin. This technique offers the advantage that the circuit has an even smaller thickness than the previous configurations, since both the processor and the condenser in DIE format occupy thicknesses ranging from 0.1 to 0.4 mm (from 0.4 mm to 0.6 mm less than the prototype presented with SMT surface-mount technology). This solution results in an almost flat surface, therefore without extrusions. Furthermore, the circuit has an extremely low cost, since the production steps are reduced compared to SMT assembly techniques.

On the other hand, the circuit has a better thermal response than plastic packages, because the thickness and consequently also the mass are considerably reduced. Furthermore, the encapsulation by a drop of resin guarantees a better thermal conductivity than plastic. Moreover, the circuit has better mechanical strength than SMT mounting, since the circuit is free of surface welds.

Advantages and Industrial Applicability of the Invention

The use of different materials with properties similar to Kapton has been tested. Among the various candidate materials to be selected as a support, the usefulness of using polypropylene in its various configurations, for example with PET, which is used in the food sector and certainly is a plastic that adapts to the application in question was found.

Polypropylene is quite rigid as a type of material. In fact it is not very elastic, although it can still have elastic properties due to the fact that the rigidity of the material is reduced. Drilling gives it greater elasticity because it reduces the mechanical resistance thereof.

Then there are also silicone materials on which printing can be performed with various silk-screen printing techniques. On an industrial level this is extremely interesting, because with silicones, screen printing configurations can be made with wire bonding printing. This technique can be performed on PET or on silicone, as can be used on Kapton. It is a technology that surely has a future to industrialize the product, because it will make the sensor ultra-thin.

With wire bonding it is possible to reach even lower thicknesses than those mentioned because it allows:
a-removing thickness, and
b-using a thermally conductive resin instead of a layer of plastic to make the covering.

This allows the thermal inertia of the transducer to be reached at a much higher speed than the configurations described above. The measurement is effective because on the one hand the thickness has decreased, and on the other hand the resin has a much greater thermal conductivity than plastic, which is now used for the silicon chip. Even if the thickness is already minimal, of the order of 0.9 mm, with the aforementioned solution, the thickness would become of the order of 0.4 mm. In this way the thermal resistance is halved.

The result is extremely surprising since as the sensor comes into contact with the skin, it reaches the body temperature instantly.

The presented solution solves the problem of the objectivity of data collection in a device that detects body clinical parameters. It has innumerable advantages:
Absence of measurement error due to positioning or alignment.
The response time of the device for detecting clinical parameters 10 is immediate, after the time of thermal inertia due to its first installation.
Absence of batteries or energy storage systems in chemical form.
The detection thermometer device is flexible and deformable, so it adapts to the surface on which it is placed.
The device is easily sanitized and recyclable.
It can be used in hospitals as it can be built in a disposable version.
The device is not invasive.
It is based on simple production processes such as FCB.
It leads to the reduction of the use of polluting raw materials such as plastic or batteries and reduced environmental impact.

The device for detecting clinical parameters 10 allows multiple and immediate measurements, without the need to prepare the instrument for measurement. In fact, as highlighted, it can store the energy transmitted by the NFC and charge a small capacitor, which allows it to perform measurements at regular time intervals. The number of measurements depends on the type of capacitor used.

The sensor stores the data of the temperature detected in a memory area, which can be read by any NCF device.

The clinical parameter detection device 10 is produced in mass and at costs that are much lower than the current ones. The device costs are very low (comparable to the cost of anti-shoplifting sensors used in supermarkets). A future production cost is estimated for a specimen that is just 0.4 Euro/pcs.

1. Detector device (10) of clinical body parameters, for the measurement and display of clinical values in instantaneous mode, in which an acquisition means (4) is integrated into a thin rigid plastic substrate (1) by placing the sensor (4) constrained on the support substrate (1) by means of an adhesive (2) which gives the device, once positioned on the measurement surface, elastic behavior and disposition to follow the curvilinear conformations on which the device is positioned adherent, the detector device (10) of clinical body parameters comprising:
i-a rigid plastic support means (1) with holes for cushioning the tangential stresses, from twisting moment, said holes being distributed alternately to solid areas configured according to a series of concentric tracks corresponding to the peripheral shape of the support (1),
ii-means (4) for the acquisition, processing and transmission of the measurements carried out in digital mode,
iii-an adhesive component (2) which fixes the acquisition, processing and transmission device (4) to the rigid plastic support (1),
iv-a patch-like outer coating component (3), which fixes the detector device (10) on the epidermis.

2. The detector device (10) of clinical body parameters, for the measurement of clinical values in instantaneous mode, according to claim 1, wherein said acquisition, processing and transmission means (4) of the measurements taken, arranged on the rigid plastic support (1) of the Kapton type, are connected to an antenna (7), also integrated in the same support substrate (1), said antenna (7) allowing the detector device (10) to interface, by wireless NFC connection, with an external display device, such NFC wireless connection ensuring the power supply of the detector device (10) itself.

3. The detector device (10) of body clinical parameters according to claim 1, wherein the hole pattern is obtained in the plane of the rectangular plastic support (1) so as to optimize its performance in terms of flexibility and elasticity to traction, the holes being distributed on a matrix according to the substrate plane spaced at regular intervals and oriented in the longitudinal and transverse directions, the arrangement leaving central areas free of holes at the components, sequentially positioned according to the longitudinal axis of symmetry, and of the printed conductor constituting the antenna (7).

4. The detector device of clinical body parameters according to claim 1, wherein the holes have a variable diameter of between 0.5 mm and 1.5 mm in relation to the thickness of the support (1) so as to reach the desired weight for the support itself.

5. The detector device of clinical body parameters according to claim 1, wherein the antenna (7) has a spiral shape, with an irregular octagonal geometry, with a conformation which maximizes the power received on the plane integral with the antenna itself and allows the sensor included in the acquisition means (4) to be read even when there is not a perfect alignment between the transmitting antenna (7) and the receiving antenna on the mobile phone, both on the axis normal to the plane and between the same planes.

6. The detector device of clinical body parameters according to claim 1, wherein the acquisition means (4) are placed in direct contact with the epidermis, since the acquisition means (4) are mounted on the outer face (a) of the a support which is in contact with the epidermis, in such a configuration of direct contact with the epidermis, a Kapton-like support material (1) with a high thermal resistance is provided.

7. The detector device of clinical body parameters according to claim 1, wherein the acquisition means (4) are placed in contact with the epidermis in an indirect manner, arranging the acquisition means (4) mounted on the face (b) opposite to the contact surface with the epidermis, providing in this condition of indirect contact the use of a support material (1), Kapton type, with low thermal resistance.

8. The detector device of clinical body parameters according to claim 1, wherein the decoupling capacitor (6) allows the decoupling of the signal part, from the power supply, the acquisition means (4) continuing to perform sampling even after the charging step of the circuit, and returning the sampled data to the next reading.

9. Method of use of the detector device of clinical body parameters according to claim 1, the method comprising the following operative steps for the relative operation:
a-the detection device (10) is supplied via the antenna (7) according to the standard NFC communication,
b-the decoupling capacitor (8) stabilizes the supply voltage of the microcontroller (4);
c-the latter makes the measurement through the sensor (9) and stores it in a memory area shared with the NFC transmitter,
d-in order to view the measurement, the data is transferred to a processing section together with the calibration data,
e-the processing section performs a correct data transformation in a temperature, for display based on a congruous linear interpolation of the calibration data.

10. The method of use of the detector device of clinical body parameters according to claim 9, wherein the detector device (10) of clinical parameters operates with a measurement time interval consisting of:
i-a first time interval which the clinical parameter detector needs to reach its thermal inertia, when it is placed the first time;
ii-a second time interval dedicated to the actual measurement,
at steady the detection device (10) faithfully and instantly tracking the body temperature with the periodic acquisition of the instantaneous sampled measurement.

11. The method of use of the detector device of clinical body parameters according to claim 10, wherein the sensor (4, 9) is mounted in DIE format with hybrid technique, being integrated into the circuit in its native form, as silicon wafer, directly on the support (1) with wire bonding technology,
with the same technique being inserted, interfaced to the processor (4), the decoupling capacitor (6),
the assembly as a whole being finally electrically insulated by means of a drop of resin.

12. The detector device of clinical body parameters according to claim 2, wherein the holes have a variable diameter of between 0.5 mm and 1.5 mm in relation to the thickness of the support (1) so as to reach the desired weight for the support itself.

13. The detector device of clinical body parameters according to claim 3, wherein the holes have a variable diameter of between 0.5 mm and 1.5 mm in relation to the thickness of the support (1) so as to reach the desired weight for the support itself.

14. The detector device of clinical body parameters according to claim 2, wherein the antenna (7) has a spiral shape, with an irregular octagonal geometry, with a conformation which maximizes the power received on the plane integral with the antenna itself and allows the sensor included in the acquisition means (4) to be read even when there is not a perfect alignment between the transmitting antenna (7) and the receiving antenna on the mobile phone, both on the axis normal to the plane and between the same planes.

15. The detector device of clinical body parameters according to claim 3, wherein the antenna (7) has a spiral shape, with an irregular octagonal geometry, with a conformation which maximizes the power received on the plane integral with the antenna itself and allows the sensor included in the acquisition means (4) to be read even when there is not a perfect alignment between the transmitting antenna (7) and the receiving antenna on the mobile phone, both on the axis normal to the plane and between the same planes.

16. The detector device of clinical body parameters according to claim 4, wherein the antenna (7) has a spiral shape, with an irregular octagonal geometry, with a conformation which maximizes the power received on the plane integral with the antenna itself and allows the sensor included in the acquisition means (4) to be read even when there is not a perfect alignment between the transmitting antenna (7) and the receiving antenna on the mobile phone, both on the axis normal to the plane and between the same planes.

17. The detector device of clinical body parameters according to claim 2, wherein the acquisition means (4) are placed in direct contact with the epidermis, since the acquisition means (4) are mounted on the outer face (a) of the a support which is in contact with the epidermis, in such a configuration of direct contact with the epidermis, a Kapton-like support material (1) with a high thermal resistance is provided.

18. The detector device of clinical body parameters according to claim 3, wherein the acquisition means (4) are placed in direct contact with the epidermis, since the acquisition means (4) are mounted on the outer face (a) of the a support which is in contact with the epidermis, in such a configuration of direct contact with the epidermis, a Kapton-like support material (1) with a high thermal resistance is provided.

19. The detector device of clinical body parameters according to claim 4, wherein the acquisition means (4) are placed in direct contact with the epidermis, since the acquisition means (4) are mounted on the outer face (a) of the a support which is in contact with the epidermis, in such a configuration of direct contact with the epidermis, a Kapton-like support material (1) with a high thermal resistance is provided.

20. The detector device of clinical body parameters according to claim 5, wherein the acquisition means (4) are placed in direct contact with the epidermis, since the acquisition means (4) are mounted on the outer face (a) of the a support which is in contact with the epidermis, in such a configuration of direct contact with the epidermis, a Kapton-like support material (1) with a high thermal resistance is provided.
